(19) United States
(12) Patent Application Publication
(10) Pub. No.: US 2026/0334942 A1
(43) Pub. Date: Oct. 29, 2026

(54) SUTURE TENSIONING DEVICE, SYSTEM AND METHOD

(71) Applicant: MOR RESEARCH APPLICATIONS LTD., Ramat Gan (IL)

(72) Inventors: Amir ORON, Ramat Gan (IL); Michael LIBRUS, Ramat Gan (IL)

(73) Assignee: MOR RESEARCH APPLICATIONS LTD., Ramat Gan (IL)

(21) Appl. No.: 19/111,611

(22) PCT Filed: Sep. 18, 2023
(86) PCT No.: PCT/IB2023/059227

§ 371 Date: Mar. 13, 2025

(30) Foreign Application Priority Data
Sep. 19, 2022 (IL) 296638

(51) Int. Cl. A61B 17/04; A61B 17/00; A61B 90/00

(52) U.S. Cl. CPC A61B 17/0483; A61B 90/06; A61B 90/36; A61B 2017/00022; A61B 2017/00221; A61B 2017/00407; A61B 2017/00438; A61B 2017/00477; A61B 2017/0496; A61B 2090/064

(57) ABSTRACT

Embodiments relate to a suture tensioning device for tensioning a suture that is operably engaged with a soft tissue repair site. In some examples, the suture tensioning device comprises a housing having a wide proximal receiving end, and a narrow distal end, wherein the housing is configured to snugly receive a digit; and a suture tensioning mechanism that is configured to fixedly receive one or more suture portions of corresponding suture ends of a suture extending from the tissue report site within the body extremity to outside the body extremity to terminate at the suture tensioning mechanism. The device may further include a suture coupling element for fixedly coupling the one or more suture portions with the tensioning mechanism; and an adjustment device configured for controllably adapting tension on the suture.

### BACKGROUND

Severed or torn tendons as well as other soft tissues including ligaments and tendons and possibly fragments of bone connected to them can cause serious pain and, in some cases, impair movement and cause disabilities. Techniques of repairing severed tendons include suturing to join the two ends of the severed tendon.

One suturing method that has established itself as one of the most widely used approaches is the Kessler repair or its modifications in which opposed severed ends of the tendon or ligament are sutured together in a particular configuration.

### BRIEF DESCRIPTION OF THE DRAWINGS

The figures illustrate generally, by way of example, but not by way of limitation, various embodiments discussed in the present document.

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. References to previously presented elements are implied without necessarily further citing the drawing or description in which they appear. The number of elements shown in the Figures should by no means be construed as limiting and is for illustrative purposes only. The figures are listed below.

FIGS. 1A-1E show various views of an upper suture tensioning device, according to some embodiments.

FIGS. 2A-2C show various views of the upper suture tensioning device operably engaging with a digit, according to some embodiments,

FIGS. 3A-3D show various views of a lower suture tensioning device, according to some embodiments.

FIG. 4 shows a lower suture tensioning device operably engaging with a digit, according to some embodiments.

### DETAILED DESCRIPTION

Postoperative complications that may be associated with the sutured joining of two ends of a severed tendon include incomplete or lack of healing of the injury due to inadequate connection of the two tendon ends, repair site gapping, local edema, peritendinous adhesion, all of which may result in physical impairment such as reduced motion or lack of grasping strength in the hand, or reduced strength and capacity in other areas in the body.

The process of tendon or ligament or soft tissue healing is attained by a biological reaction wherein the cells in the injured area act to repair the injured tissue while making a matrix of tissue. Approximation of injured tissues is key for proper healing and the transfer of force as well as excursion of tissue may act to enhance these processes. Moreover, correct and optimal placement of the suturing material and assurance of optimal alignment and contact of injured tendon or ligament ends is key for timely and optimal healing.

Since impaired mobility and/or reduced grasping force may not always necessarily directly be associated with reduced suture tensioning but relate to, for example, local edema and/or peritendinous adhesions, repeated measurement of the tension force may provide an indication on whether impaired mobility and/or reduced grasping is due to reduced suture tensioning or not.

Embodiments pertain to suture tensioning devices which are configured to postoperatively measure the magnitude of tension force applied on the sutures that are coupled with repair sites of soft tissue such as tendons during patient rehabilitation for objectively assessing whether the repair procedure has been successful and for detecting postoperative complications.

Embodiments of the suture tensioning devices allow for feedback-based suture tensioning and are, for example, configured to enable incremental tensioning of the suture to enhance the internal healing of the tendon (also: intrinsic healing), to decelerate external healing, which may manifest itself in decreased external scarring.

In some examples, the suture tensioning devices may be employed for determining whether suture filament re-tensioning is required. The suture filament tensioning devices may be configured to allow suture filament re-tensioning under a selected imaging modality (e.g., Ultrasound imaging) while providing tension force feedback. This way, gapping reduction can be associated with the tension force that needs to be applied to arrive at the desired reduction in gap.

According to some embodiments, a suture tensioning device may include a housing, e.g., in the form of a hollow socket having a wide proximal receiving end for receiving a body part, and a narrow distal end. The housing may be configured to receive a body part which may be an extremity such as a portion of digit having a main longitudinal axis. The housing may be configured to be at least partially applicable around or fittingly engage with the extremity. The extremity may be of a mammal such as a human.

In some examples, the socket may be a partially open socket having an arched, open cross-section. The socket's narrow distal end may be configured to form-fittingly constrain axial movement of the extremity in distal direction. In some examples, the narrow distal end may be closed end shaped in the form of a cap, e.g., having an about circular cross section or an arch-shaped cross section

In the context of the present disclosure, without limiting, the term “distal” is to indicate an end of the device that is situated further away from a soft tissue repair site, and the term “proximal” is an end of the device that is situated closer to the soft tissue repair site, e.g., during normal use of the suture tensioning device.

The suture tensioning device may further include a suture tensioning mechanism. The suture tensioning mechanism may be configured to fixedly receive at least one distal end of a suture. The suture may extend from the soft tissue repair site (e.g., tendon repair site) within the patient's body, exit the patient's extremity, for example, via the distal tip or from any region where there is enough soft tissue to surround the suture filament leaving the body, of the extremity (e.g., the distal tip of a patient's digit), and terminate outside the patient's body at the suture tensioning mechanism.

In some embodiments, suture tensioning device may include a suture coupling element (e.g., suture snare, through-hole) for fixedly coupling a distal portion of the suture filament with the tensioning mechanism. In some examples, the suture coupling element may be part of the suture tensioning mechanism.

The suture tensioning mechanism may further include an adjustment device that allows for controllably tensioning the suture filament by applying a tensioning force in distal direction with respect to the soft tissue repair site. The adjustment device may include an actuator such as a wheel, a knob or slider that is operably engageable by a user of the suture tensioning device and which allows to selectively increase or decrease tension that is applied to the suture. The user may be a medical professional or the patient.

In some embodiments, the adjustment device may be configured to selectively allow incremental, stepwise increase or decrease of the applied tensioning force. For example, the adjustment device may include an actuator having a serrated surface formed by a plurality of neighboring teeth, and a flexible tab that is configured to project into a detent between each two neighboring teeth. The flexible tab is configured to tend to restore a nominal positional relationship with respect to the teeth, and allows to elastically deflect in at least one direction responsive to the movement of the teeth relative to the tab.

In some embodiments, the actuator of the adjustment device may be configured as a cogwheel. In some other embodiments, the actuator of the adjustment device may be configured as a rack.

In some embodiments, the adjustment device may be configured as a ratchet configured for increasing, by default, tensioning of the suture, and prevent unwanted decrease in suture tensioning. For example, the actuator may include a cogged wheel having teeth and a biased pawl configured to engage with the teeth of the cogged wheel. The wheel and the pawl are configured such that the wheel may only be rotatable in a first direction to effect tensioning of the suture.

The suture tensioning mechanism may include a tensiometer that is operably coupled with the suture and/or the adjustment device. The tensiometer may be implemented electronically and/or mechanically. For example, the tensiometer may include a mechanical energy storage device configured to gauge suture tension. The tensiometer may provide an output indicative of the measured tension to which the suture is subjected to.

The soft tissue repair site may be characterized based on the gauged or sensed suture tension applied on the suture.

In some embodiments, the suture tensioning device may include a communication module that is configured to output (e.g., transmit in a wired or wireless manner) signals that encode data relating to sensed suture tension to one or more computerized platforms. A computerized platform may include a multifunction mobile communication device also known as “smartphone”, a personal computer, a laptop computer, a tablet computer, a server (which may relate to one or more servers or storage systems and/or services associated with a business or corporate entity, including for example, a file hosting service, cloud storage service, online file storage provider, peer-to-peer file storage or hosting service and/or a cyberlocker), personal digital assistant, a workstation, a wearable device, a handheld computer, and/or a notebook computer.

In some embodiments, the device and/or the system may be configured to delimit the tension force to which the suture can be subjected to avoid excess tensioning of the suture, to avoid the application of excess force on the tissue repair site.

In some embodiments, the device and/or the system may be configured to record or log, along with a time stamp, the tension force to which the suture is subjected to, optionally along with clinical information relating to grasping force, span of motion, and/or the like.

This may be performed both by a professional guiding and determining the desired time, repetition rate, repetition number their manner and their timing, The device and/or system may allow for targeting the desired values during correct rehabilitation and later assuring the patient adheres to the desired rehabilitation protocol-when performing with or without supervision. In some examples, the system may include an output (e.g., one or more visual and/or auditory outputs) for providing the user with an indication concerning the rehabilitation progress and/or adherence.

It is known to the art that as tendon or tissue healing ensues the forces acting on the suture itself may diminish. The data accrued by the different cases will be collected and used for assessing changes and differences in strength accrual of the repair within the same individual and between individuals and between digits as to allow for interpretation of these findings and for building and differentiating rehabilitation protocols tailor made for certain patients. For example a hastened rehabilitation program for a young, non-smoking professional sportsman and a halted, slower rehabilitation for an old, unhealthy smoker.

Although embodiments of the device are disclosed with respect to operable engagement with digits, this should by no means be construed in a limiting. Accordingly, embodiments of the device may be also be operably engageable with respect to Achilles tendon, patellar tendon and/or other soft tissues including ligaments and/or tendons and possibly fragments of bone connected to them can cause serious pain and, in some cases, impair movement and cause disabilities.

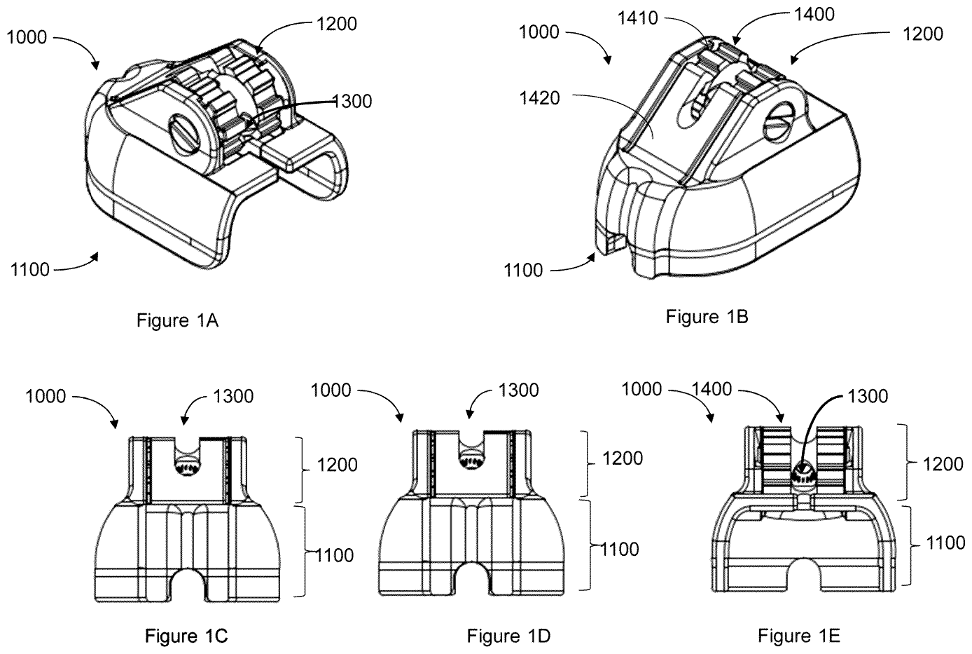

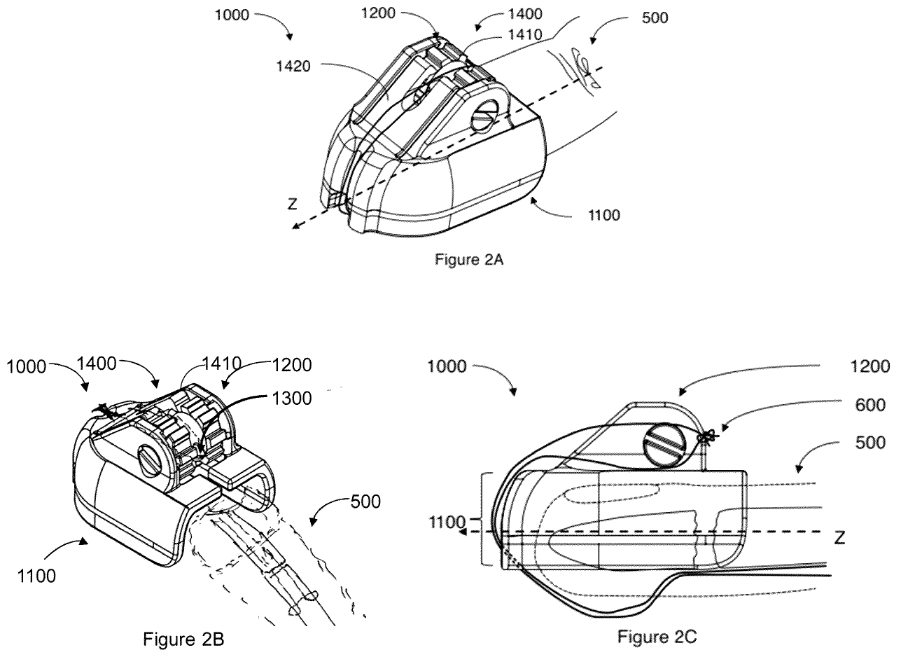

Reference is now made to FIGS. 1A-1E and to FIGS. 2A-2C. In some embodiments, an upper suture tensioning device 1000 may include an upper housing 1100, e.g., in the form of a hollow socket having wide proximal receiving end for a body part, and a narrow distal end. Upper housing 1100 may be configured to receive a body part which may be an extremity such as a portion of digit 500 having a longitudinal axis Z. The term “upper” refers to a configuration in which upper housing 1100 posteriorly engages with digit 500.

In some examples, upper housing 1100 may be a partially open socket having an arched, open cross-section. The socket's narrow distal end may be configured to form-fittingly constrain axial movement of the extremity in distal direction. In some examples, the narrow distal end may be closed end shaped in the form of a cap, e.g., having an about circular cross section or an arch-shaped cross section

Upper suture tensioning device 1000 may further include an upper suture tensioning mechanism 1200. Upper suture tensioning mechanism 1200 may be configured to fixedly receive at least one distal end of a suture 600. Suture 600 may extend from the soft tissue repair site (e.g., tendon repair site) within the patient's body, exit the patient's extremity via the distal tip of the extremity (e. g,, the distal tip of a patient's digit) and terminate outside the patient's body at upper suture tensioning mechanism 1200.

In some embodiments, upper suture tensioning mechanism 1200 may include a suture coupling element 1300 for fixedly coupling a distal portion of the suture with the tensioning mechanism.

Upper Suture tensioning mechanism 1200 may further include an adjustment device 1400 that allows for controllably tensioning the suture by applying a tensioning force in distal direction with respect to the soft tissue repair site.

Adjustment device 1400 may include at least one actuator 1410 such as a wheel, a knob or slider that is operably engageable by a user of suture tensioning device 1000, and an elastic deflectable tab 1420 that operably engages with actuator 1410 such to allow selectively increasing or decreasing, e.g., incrementally, a tension force that is applied to the suture. In some examples, adjustment device 1400 may include a plurality of actuators configured such that each wheel can be rotated independent from the other wheel to selectively pull different ends of the suture.

In the discussion that follows, without being construed as limiting, actuator is shown to be implemented as a wheel.

In some embodiments, actuator 1410 may include a cogged wheel having teeth and deflectable tab 1420 configured to engage with each detent formed by two neighboring teeth of the cogged wheel. In some examples, the wheel and the pawl are configured such that actuator 1410 may only be rotatable in a first direction to effect tensioning of the suture, thereby implementing a ratchet configuration.

In some embodiments, suture coupling element 1300 may be implemented as a through-hole extending through wheel 1400.

In some embodiments, adjustment device 1400 may be configured as a ratchet configured for increasing, by default, tensioning of the suture, and prevent unwanted decrease in suture tensioning. For example, the actuator may include a cogged wheel having teeth and a deflectable tab configured to engage with the teeth of the cogged wheel. The wheel and the pawl are configured such that the wheel may only be rotatable in a first direction to effect tensioning of the suture.

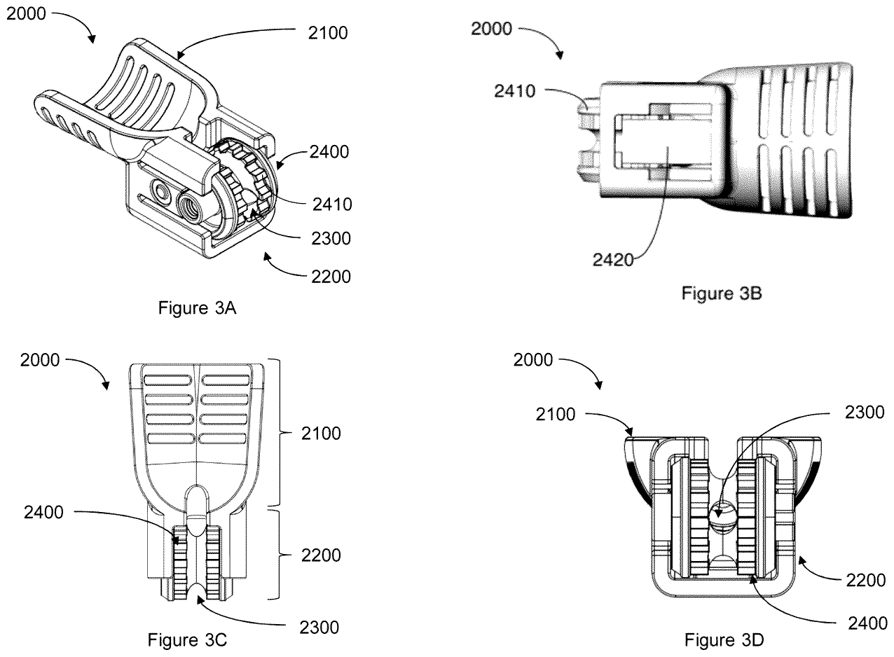

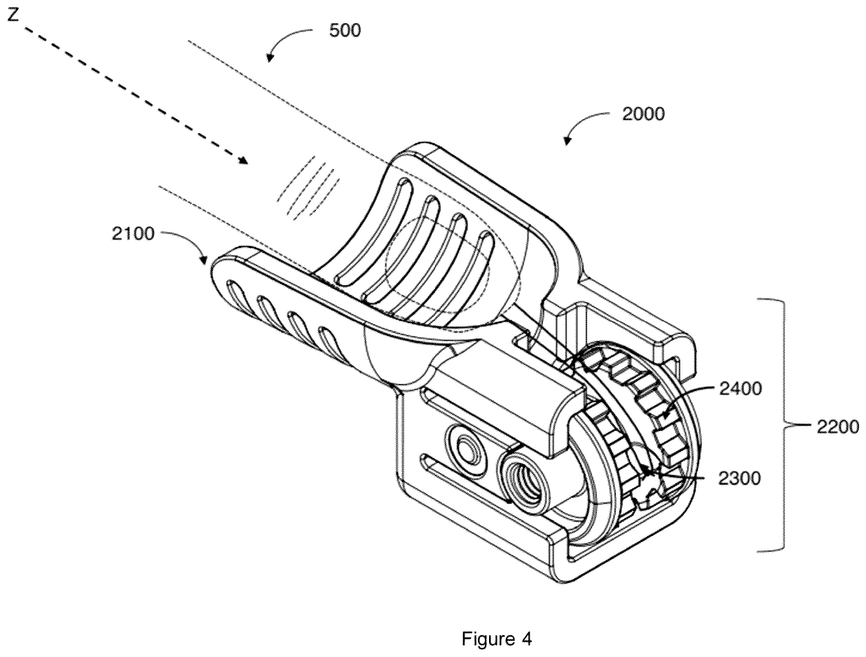

Reference is now made to FIGS. 3A-3D and to FIG. 4. In some embodiments, a lower suture tensioning device 2000 may include a lower housing 2100, e.g., in the form of a hollow socket having wide proximal receiving end for a body part, and a narrow distal end. Lower housing 2100 may be configured to receive a body part which may be an extremity such as a portion of digit 500 having a longitudinal axis Z. The term “lower” refers to a configuration in which lower housing 2100 anteriorly engages with digit 500.

In some examples, lower housing 2100 may be a partially open socket having an arched, open cross-section. The socket's narrow distal end may be configured to form-fittingly constrain axial movement of the extremity in distal direction. In some examples, the narrow distal end may be closed end shaped in the form of a cap, e.g., having an about circular cross section or an arch-shaped cross section

Lower suture tensioning device 2000 may further include a lower suture tensioning mechanism 2200. Lower suture tensioning mechanism 2200 may be configured to fixedly receive at least one distal end of a suture 600. Suture 600 may extend from the soft tissue repair site (e.g., tendon repair site) within the patient's body, exit the patient's extremity via the distal tip of the extremity (e.g., the distal tip of a patient's digit) and terminate outside the patient's body at lower suture tensioning mechanism 2200.

In some embodiments, lower suture tensioning mechanism 2200 may include a suture coupling element 2300 (e.g., through-whole, suture snare) for fixedly coupling a distal portion of the suture with the tensioning mechanism.

Lower suture tensioning mechanism 2200 may further include an adjustment device 2400 that allows for controllably tensioning the suture by applying a tensioning force in distal direction with respect to the soft tissue repair site.

In some embodiments, adjustment device 2400 may include an actuator 2410 such as a wheel, a knob or slider, and an elastic deflectable tab 2420 that operably engages with actuator 1410 such to allow selectively increasing or decreasing, e.g., incrementally, a tension force that is applied to the suture.

In some embodiments, suture coupling element 1300 may be implemented as a through-hole extending through wheel 1400.

### Additional Examples

Example 1 concerns a suture tensioning device for tensioning a suture that is operably engaged with a soft tissue repair site, the suture tensioning device comprising:
a housing having a wide proximal receiving end, and a narrow distal end, wherein the housing is configured to snugly receive a digit;
a suture tensioning mechanism that is configured to fixedly receive one or more suture portions of corresponding suture ends of a suture extending from the tissue report site within the body extremity to outside the body extremity to terminate at the suture tensioning mechanism;
a suture coupling element for fixedly coupling the one or more suture portions with the tensioning mechanism; and
an adjustment device configured for controllably adapting tension on the suture.

Example 2 includes the subject matter of Example 1 and, optionally, wherein the adjustment device comprises an actuator that is operably engageable by a user of the suture tensioning device and which allows to selectively increase or decrease tension that is applied to the suture.

Example 3 includes the subject matter of any one or more of the examples 1 to 2 and, optionally, wherein the adjustment device is configured as a ratchet for tensioning of the suture, and to prevent unwanted decrease in suture tensioning.

Example 4 includes the subject matter of any one or more of the examples 2 to 3 and, optionally, wherein the tensioning mechanism comprises:
a cogged wheel having teeth; and
a biased pawl configured to engage with the teeth of the cogged wheel,
wherein the wheel and the pawl are configured such that the wheel is only be rotatable, by default, in one direction.

Example 5 includes the subject matter of any one or more of the examples 1 to 4 and, optionally, a tensiometer that is operably coupled with the suture and/or the adjustment device for providing an output indicative of a tension force applied on the suture.

Example 6 includes the subject matter of Example 5 and, optionally, a communication module that is configured to output signals which encode data relating to suture tension sensed by the tensiometer to one or more computerized platforms.

Example 7 includes a suture tensioning device of any one or more of the preceding claims, including a belt or strap that is externally couplable with body part of a patient and to which suture filaments emanating from a suture site can be connected, the suture tensioning device further comprising:
a suture tensioning mechanism that is configured to fixedly receive one or more suture portions of corresponding suture ends of a suture extending from the tissue report site within the body extremity to outside the body extremity to terminate at the suture tensioning mechanism;
a suture coupling element for fixedly coupling the one or more suture portions with the tensioning mechanism; and
an adjustment device configured for controllably adapting tension on the suture.

Example 8 includes the subject matter of example 7 and, optionally, wherein the adjustment device comprises an actuator that is operably engageable by a user of the suture tensioning device and which allows to selectively increase or decrease tension that is applied to the suture.

Example 9 includes the subject matter of or more of the examples 7 to 8 and, optionally, wherein the adjustment device is configured as a ratchet for tensioning of the suture, and to prevent unwanted decrease in suture tensioning.

Example 10 includes the subject matter of suture tensioning device of any one or more of the examples 7 to 9 and, optionally, wherein tensioning mechanism comprises:
a cogged wheel having teeth; and
a biased pawl configured to engage with the teeth of the cogged wheel,
wherein the wheel and the pawl are configured such that the wheel is only be rotatable, by default, in one direction.

Example 11 includes the subject matter of any one or more of the Examples 7 to 10 and, optionally, further comprising:
a tensiometer that is operably coupled with the suture and/or the adjustment device for providing an output indicative of a tension force applied on the suture.

Example 12 includes the subject matter of Example 11 and, optionally,
a communication module that is configured to output signals which encode data relating to suture tension sensed by the tensiometer to one or more computerized platforms.

Example 13 concerns a suture tensioning monitoring and assessment system, comprising:
a suture tensioning device
one or more computerized platforms configured to receive signals encoding data relating to suture tension sensed by a tensiometer.

Example 14 includes the subject matter of Example 13 and, optionally, an imaging apparatus for imaging the suture repair site for determining one or more characteristics of the soft tissue repair site, and
wherein the system is configured to determine, based on the one or more characteristics of the soft tissue repair site, one or more adjustment parameter values of the displacement device.

Example 15 includes the subject matter of Example 14 and, optionally, a motion drive configured for adjusting the one or more actuators of the displacement device based on the one or more adjustment parameter values.

Example 16 includes the subject matte of example 15 and, optionally, wherein the one or more actuators are automatically, semi-automatically or manually adjustable through the motion drive.

Example 17 concerns a method for repairing severed soft tissue such as a tendon of an extremity of a mammal, the method comprising:
coupling opposite ends of the severed soft tissue using a suture in a manner such that the ends of the suture are outside the body of the mammal;
operably engaging the extremity with a suture tensioning device;
operably coupling the suture ends with the suture tensioning device such to allow tensioning, with the device, the suture; and
recording the tension force applied on the suture.

In some embodiments, the adjustment device may be configured as a ratchet configured for increasing, by default, tensioning of the suture, and prevent unwanted decrease in suture tensioning. For example, the actuator may include a cogged wheel having teeth and a biased pawl configured to engage with the teeth of the cogged wheel. The wheel and the pawl are configured such that the wheel may only be rotatable in a first direction to effect tensioning of the suture.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

The various features and steps discussed above, as well as other known equivalents for each such feature or step, can be mixed and matched by one of ordinary skill in this art to perform methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

In the discussion, unless otherwise stated, adjectives such as “substantially” and “about” that modify a condition or relationship characteristic of a feature or features of an embodiment of the apparatus, are to be understood to mean that the condition or characteristic is defined to within tolerances that are acceptable for operation of the embodiment for an application for which it is intended.

Unless otherwise specified, the terms ‘about’ and/or ‘close’ with respect to a magnitude or a numerical value may imply to be within an inclusive range of −10% to +10% of the respective magnitude or value.

Unless otherwise specified, the terms ‘about’ or ‘close’ imply at or in a region of, or close to a location or a part of an object relative to other parts or regions of the object.

Positional terms such as “upper”, “lower” “right”, “left”, “bottom”, “below”, “lowered”, “low”, “top”, “above”, “elevated”, “high”, “vertical” and “horizontal” as well as grammatical variations thereof as may be used herein do not necessarily indicate that, for example, a “bottom” component is below a “top” component, or that a component that is “below” is indeed “below” another component or that a component that is “above” is indeed “above” another component as such directions, components or both may be flipped, rotated, moved in space, placed in a diagonal orientation or position, placed horizontally or vertically, or similarly modified. Accordingly, it will be appreciated that the terms “bottom”, “below”, “top” and “above” may be used herein for exemplary purposes only, to illustrate the relative positioning or placement of certain components, to indicate a first and a second component or to do both.

“Coupled with” means indirectly or directly “coupled with”.

As used herein, unless otherwise specified, the use of the ordinal adjectives “first”, “second”, etc., to describe like objects, merely indicate that different instances of like objects are being referred to and are not intended to imply that the objects so described must be in a given sequence, temporally, in ranking, and/or in any other manner.

The descriptions of the various embodiments of the present apparatus have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A suture tensioning device for tensioning a suture that is operably engaged with a soft tissue repair site, the suture tensioning device comprising:
a housing having a wide proximal receiving end, and a narrow distal end, wherein the housing is configured to snugly receive a digit;
a suture tensioning mechanism that is configured to fixedly receive one or more suture portions of corresponding suture ends of a suture extending from the tissue report site within the body extremity to outside the body extremity to terminate at the suture tensioning mechanism;
a suture coupling element for fixedly coupling the one or more suture portions with the tensioning mechanism; and
an adjustment device configured for controllably adapting tension on the suture.

2. The suture tensioning device of claim 1, wherein the adjustment device comprises an actuator that is operably engageable by a user of the suture tensioning device and which allows to selectively increase or decrease tension that is applied to the suture.

3. The suture tensioning device of claim 1, wherein the adjustment device is configured as a ratchet for tensioning of the suture, and to prevent unwanted decrease in suture tensioning.

4. The suture tensioning device of claim 2, wherein tensioning mechanism comprises:
a cogged wheel having teeth; and
a biased pawl configured to engage with the teeth of the cogged wheel,
wherein the wheel and the pawl are configured such that the wheel is only be rotatable, by default, in one direction.

5. The suture tensioning device of claim 1, further comprising: a tensiometer that is operably coupled with the suture and/or the adjustment device for providing an output indicative of a tension force applied on the suture.

6. The suture tensioning device of claim 5, further comprising:
a communication module that is configured to output signals which encode data relating to suture tension sensed by the tensiometer to one or more computerized platforms.

7. A suture tensioning monitoring and assessment system, comprising:
a housing having a wide proximal receiving end, and a narrow distal end, wherein the housing is configured to snugly receive a digit;
a suture tensioning mechanism that is configured to fixedly receive one or more suture portions of corresponding suture ends of a suture extending from the tissue report site within the body extremity to outside the body extremity to terminate at the suture tensioning mechanism;
a suture coupling element for fixedly coupling the one or more suture portions with the tensioning mechanism; and
an adjustment device configured for controllably adapting tension on the suture
a tensiometer that is operably coupled with the suture and/or the adjustment device for providing an output indicative of a tension force applied on the suture; and one or more computerized platforms configured to receive signals encoding data relating to suture tension sensed by the tensiometer.

8. The suture tensioning monitoring and assessment system of claim 7, further comprising an imaging apparatus for imaging the suture repair site for determining one or more characteristics of the soft tissue repair site, and
wherein the system is configured to determine, based on the one or more characteristics of the soft tissue repair site, one or more adjustment parameter values of the displacement device.

9. The suture tensioning monitoring and assessment system of claim 8, comprising a motion drive configured for adjusting the one or more actuators of the displacement device based on the adjustment parameter values.

10. The suture tensioning monitoring and assessment system of claim 9, wherein the one or more actuators are automatically, semi-automatically or manually adjustable through the motion drive.

11. A suture tensioning device comprising:
a belt or strap that is externally couplable with body part of a patient and to which suture filaments emanating from a suture site can be connected, the suture tensioning device further comprising:
a suture tensioning mechanism that is configured to fixedly receive one or more suture portions of corresponding suture ends of a suture extending from the tissue report site within the body extremity to outside the body extremity to terminate at the suture tensioning mechanism;
a suture coupling element for fixedly coupling the one or more suture portions with the tensioning mechanism; and
an adjustment device configured for controllably adapting tension on the suture.

12. The suture tensioning device of claim 11, wherein the adjustment device comprises an actuator that is operably engageable by a user of the suture tensioning device and which allows to selectively increase or decrease tension that is applied to the suture.

13. The suture tensioning device of claim 11, wherein the adjustment device is configured as a ratchet for tensioning of the suture, and to prevent unwanted decrease in suture tensioning.

14. The suture tensioning device of any one or more of the claim 11, wherein tensioning mechanism comprises:
a cogged wheel having teeth; and
a biased pawl configured to engage with the teeth of the cogged wheel,
wherein the wheel and the pawl are configured such that the wheel is only be rotatable, by default, in one direction.

15. The suture tensioning device of claim 11, further comprising:
a tensiometer that is operably coupled with the suture and/or the adjustment device for providing an output indicative of a tension force applied on the suture.

16. The suture tensioning device of claim 15, further comprising:
a communication module that is configured to output signals which encode data relating to suture tension sensed by the tensiometer to one or more computerized platforms.

17. A method for repairing severed soft tissue such as a tendon of an extremity of a mammal, the method comprising:
coupling opposite ends of the severed soft tissue using a suture in a manner such that the ends of the suture are outside the body of the mammal;
operably engaging the extremity with a suture tensioning device;
operably coupling the suture ends with the suture tensioning device such to allow tensioning, with the device, the suture; and
recording the tension force applied on the suture.

18. The method of claim 17, comprising selectively increasing or decreasing tension that is applied to the suture.

19. The method of claim 18, comprising outputting signals which encode data relating to suture tension.

20. The method of claim 18, comprising employing a ratchet for tensioning of the suture, and to prevent unwanted decrease in suture tensioning.
